(19) United States
(12) Patent Application Publication
(10) Pub. No.: US 2023/0083117 A1
(43) Pub. Date: Mar. 16, 2023

(54) USE OF A COMBINATION OF NITROGEN AND HYDROGEN BASED COMPOUNDS WITH AN AMINO ACID AND A STIMULANT

(71) Applicant: Eric Marchewitz, ake Orion, MI (US)

(72) Inventor: Eric Marchewitz, ake Orion, MI (US)

(21) Appl. No.: 17/945,667

(22) Filed: Sep. 15, 2022

Related U.S. Application Data
(60) Provisional application No. 63/244,525, filed on Sep. 15, 2021.
(60) Provisional application No. 63/247,338, filed on Sep. 23, 2021.

(51) Int. Cl. A61K 33/00; A61K 31/198; A61K 31/205; A61K 31/522; A61K 31/137; A61K 31/131; A61K 9/00

(52) U.S. Cl. CPC A61K 33/00; A61K 31/198; A61K 31/205; A61K 31/522; A61K 31/137; A61K 31/131; A61K 9/0053

(57) ABSTRACT

This invention relates a method of orally administering a liquid consisting of a combination of nitrogen based compounds consisting of nitrogen, nitric oxide or nitrous oxide with amino acids and/or amino acid peptides salts and esters and stimulants and stimulant salts to increase athletic performance, anti-aging focus, and energy output, and a method of administering a combination of combining hydrogen compounds, consisting of hydrogen gases and/or ions including but not limited to H2 and H+ signaling molecules with both D and L forms of amino acids and/or amino acid peptides salts and esters, and stimulants and stimulant salts for providing increased athletic performance via hydration, for increased energy output, and for the reduction of free radical damage.

### FIELD OF THE INVENTION

This invention relates a method of orally administering a liquid consisting of a combination of nitrogen based compounds consisting of nitrogen, nitric oxide or nitrous oxide with amino acids and/or amino acid peptides salts and esters and stimulants and stimulant salts to increase athletic performance, anti-aging focus, and energy output, and a method of administering a combination of combining hydrogen compounds, consisting of hydrogen gases and/or ions including but not limited to H2 and H+ signaling molecules with both D and L forms of amino acids and/or amino acid peptides salts and esters, and stimulants and stimulant salts for providing increased athletic performance via hydration, for increased energy output, and for the reduction of free radical damage.

### BACKGROUND OF THE INVENTION

Nitrogen donors and signaling molecules such as nitrous oxide are useful in potentiating repair, focus, performance, and recovery from activities such as exercise. Nitrogen signaling molecules represent a major way to increase athletic performance and energy output as well as increased focus with aging. Hydrogen donors and signaling molecules such as H2 and H− are useful antioxidant for anti-aging and reduction of reactive oxygen species. By combining nitrogen or hydrogen signaling molecules with both D and L forms of amino acids and/or stimulants the disclosures set forth below allow for an oral supplement that can achieve these desired results. There exists a need for a nitrogen based oral liquid combined with amino acids and/or stimulants for the purpose of increasing athletic performance and energy output as well as increased focus with aging. There also exists a need for a hydrogen based oral liquid combined with amino acids and/or stimulants for the purpose of increasing athletic performance via hydration, to reduce free radicals, and to increase energy output.

Disclosed in this invention is a series of methods for combining a nitrogen based liquid with amino acids and/or stimulants, for the purpose of increasing athletic performance and energy output as well as increased focus with aging. Also disclosed is a series of methods for combining a hydrogen based oral liquid with amino acids and/or stimulants for the purpose of increasing athletic performance via hydration, to reduce free radicals, and to increase energy output.

### SUMMARY OF THE INVENTION

In accordance with a first aspect, methods are disclosed for combining a nitrogen based liquid with amino acids and/or stimulants for the purpose of increasing athletic performance and energy output as well as increased focus with aging. Methods are also disclosed for combining hydrogen based liquid with amino acids and/or stimulants for the purpose of increasing athletic performance via hydration, to reduce free radicals, and to increase energy output.

From the foregoing disclosure and the detailed description set forth below of various preferred embodiments of the invention it will be apparent to those skilled in the art that the disclosed invention provides a significant advance in the methods of administering nitrogen and/or hydrogen based compounds in a liquid oral supplement with an Amino Acid and a stimulant. The unique formulas disclosed herein will provide a substantial advantage over traditional oral administration of energy dietary supplements. Additional features and advantages of various preferred embodiments will be better understood in view of the detailed description provided below.

### DETAILED DESCRIPTION

It will be apparent to those skilled in the art, that is, to those who have a knowledge or experience in this area of technology that many variations are possible for the method of orally administering a combination of combining nitrogen compounds, consisting of Nitrogen N2, Nitric Oxide NO and/or Nitrous Oxide N2O2 signaling molecules with both D and L forms of amino acids and/or amino acid peptides salts and esters, and stimulants and stimulant salts. The D and L forms of Amino acids might include but are not limited to one or more of the following:

Citrulline—ergogenic, Leucine—anabolic, Proline—wound healing, Theanine—neurotransmitter support, and Carnitine—cellular energy. Additional Amino Acids which might be utilized include but are not limited to one or more of the following:

### Arginine

### Agmatine

### Lucine

### Isolucine

### Valine

### NorValine

### Taurine

### Creatine

### Phenylalanine

### Tyrosine

### Glycine

### Alanine

### Beta-Alanine

### Histadine

### Glutamine

### Ornithine

### Betaine

The stimulants and stimulant salts might include, but are not limited to caffeine, caffeine malate, synephrine, methyl-synephrine, and DMAA. The methods and formulations disclosed herein are new and have a distinct advantage over existing methods of drugs and/or dietary supplements for providing increased athletic performance and increased energy output.

It will also be apparent to those skilled in the art, that is, to those who have a knowledge or experience in this area of technology that many variations are possible for the method of orally administering a combination of combining hydrogen compounds, consisting of hydrogen gases and/or ions including but not limited to H2 and H+ signaling molecules with both D and L forms of amino acids and/or amino acid peptides salts and esters, and stimulants and stimulant salts. The D and L forms of Amino acids might include but are not limited to one or more of the following:

Citrulline—ergogenic,
Leucine—anabolic,
Proline—wound healing,
Theanine—neurotransmitter support, and
Carnitine—cellular energy. Additional Amino Acids which might be utilized include but are not limited to one or more of the following:

### Arginine

### Agmatine

### Lucine

### Isolucine

### Valine

### NorValine

### Taurine

### Creatine

### Phenylalanine

### Tyrosine

### Glycine

### Alanine

### Beta-Alanine

### Histadine

### Glutamine

### Ornithine

### Betaine

The stimulants and stimulant salts might include, but are not limited to caffeine, caffeine malate, synephrine, methyl-synephrine, and DMAA. The methods and formulations disclosed herein are new and have a distinct advantage over existing methods of drugs and/or dietary supplements for providing increased athletic performance via hydration, for increased energy output, and for the reduction of free radical damage.

All of the compound formulations disclosed herein would be administered orally within a liquid carrier. Alternatively, the formulations might be administered via IV, topically, or sublingually. In the preferred embodiment the nitrogen and hydrogen compounds would be combined with both an amino acid and a stimulant. In an alternative embodiment the nitrogen and hydrogen compounds would be combined with only an amino acid. In another alternative embodiment the nitrogen and hydrogen compounds would be combined with only a stimulant. In the preferred embodiment these various compounds would preferably be administered orally mixed with a liquid carrier in appropriate unit doses.

The preferred amount of each of the three active ingredients within the nitrogen based formulation that is to be orally administered would depend on various factors such as the specific drug or dietary supplement that needs to be utilized to obtain the desired result. The concentration of the nitrogen compound in the formulation might be between 0.1% and 1%. The preferred concentration would be between 0.3% and 0.5%. The concentration of the Amino Acid in the formulation might be between 0.2% and 0.4%. It might be between 0.01% and 3%. The preferred concentration would be between 0.1% and 0.4%. The concentration of the stimulant in the formulation might be 5 between 0.1% and 0.4%. It might be between 0.2% and 0.3%. The preferred concentration would be between 0.1% and 0.3%.

The preferred amount of each of the three active ingredients within the hydrogen based formulation that is to be orally administered would depend on various factors such as the specific drug or dietary supplement that needs to be utilized to obtain the desired result. The concentration of the hydrogen compound in the formulation might be between 0.1% and 1%. The preferred concentration would be between 0.3% and 0.5%. The concentration of the Amino Acid in the formulation might be between 0.2% and 0.4%. It might be between 0.01% and 3%. The preferred concentration would be between 0.1% and 0.4%. The concentration of the stimulant in the formulation might be 5 between 0.1% and 0.4%. It might be between 0.2% and 0.3%. The preferred concentration would be between 0.1% and 0.3%.

Example Nitrogen Based Formulation One:

A drink made with water, and a sweetener consisting of:

### Aceulfame K

### Sucralose

### Stevia

### Monk Fruit Extract

### Erythritol

### High Fructose Corn Syrup

### Sugar

### Example Nitrogen Based Formulation Two:

A drink containing 100 mg caffeine anhydrous, an amino acid salt such as citrulline malate and a sweetener such as sucralose along with nitrous oxide for the nitrogen doner.

### Example Nitrogen Based Formulation Three:

A drink containing 200 mg caffeine malate, a group of amino acids such as 100 mg of each: agamatine sulfate, L-lucine, and L-taurine plus a sweetener such as stevia leaf extract and finally a nitrogen doner gas such as nitric oxide.

### Example Hydrogen Based Formulation One:

A drink made with water, and a sweetener consisting of:

### Aceulfame K

### Sucralose

### Stevia

### Monk Fruit Extract

### Erythritol

### High Fructose Corn Syrup

### Sugar

### Example Hydrogen Based Formulation Two:

A drink containing 100 mg caffeine anhydrous, an amino acid salt such as citrulline malate and a sweetener such as sucralose along with H+ for the hydrogen doner.

### Example Hydrogen Based Formulation Three:

A drink containing 200 mg caffeine malate, a group of amino acids such as 100 mg of each: agamatine sulfate, L-lucine, and L-taurine plus a sweetener such as stevia leaf extract and finally a hydrogen doner gas such as H−.

From the foregoing disclosure and detailed description of certain preferred embodiments, it will be apparent that various modifications, additions, and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method of orally administering a liquid consisting of a combination of nitrogen based compounds consisting of Nitrogen, Nitric Oxide or Nitrous Oxide with amino acids and/or amino acid peptides salts and esters and stimulants and stimulant salts.

2. The method of claim 1 wherein the Amino Acid consists of at least one or more of a Citrulline, Leucine, Proline, Theanine, and Carnitine.

3. The method of claim 1 wherein the stimulant consists of at least one or more of caffeine, caffeine malate, synephrine, methyl-synephrine, and DMAA.

4. A method of administering a combination of combining hydrogen based compounds, consisting of hydrogen gases and/or ions including but not limited to H2 and H+ signaling molecules with both D and L forms of amino acids and/or amino acid peptides salts and esters, and stimulants and stimulant salts.

5. The method of claim 4 wherein the Amino Acid consists of at least one or more of a Citrulline, Leucine, Proline, Theanine, and Carnitine.

6. The method of claim 4 wherein the stimulant consists of at least one or more of caffeine, caffeine malate, synephrine, methyl-synephrine, and DMAA.
